(19) United States
(12) Patent Application Publication
(10) Pub. No.: US 2014/0056941 A1
(43) Pub. Date: Feb. 27, 2014

(54) ANTIGENIC FORMULATION

(71) Applicant: Erwin Mombarg, Cuijk (NL)

(72) Inventor: Erwin Mombarg, Cuijk (NL)

(21) Appl. No.: 14/112,557

(22) PCT Filed: Apr. 20, 2012
(86) PCT No.: PCT/EP2012/057231

§ 371 Date: Oct. 18, 2013

Related U.S. Application Data
(60) Provisional application No. 61/479,007, filed on Apr. 26, 2011.

(30) Foreign Application Priority Data
Apr. 22, 2011 (EP) 11163570.2

(51) Int. Cl. A61K 39/39

(52) U.S. Cl. CPC A61K 39/39
USPC 424/211.1; 424/184.1; 424/236.1

(57) ABSTRACT

The present invention pertains to an antigenic formulation comprising a biological antigen, wherein the formulation comprises an oil containing as a principle constituent a fatty acid ester of eleostearic acid. The invention also pertains to the use of the said oil to manufacture an antigenic formulation.

The present inventions pertains to an antigenic formulation comprising at least one biological antigen.

Formulations comprising biological antigens are known in the art and may for example be used for diagnostic purposes or as vaccines to prevent, mitigate or cure a disease caused by a (micro)organism such as bacteria, viruses, rickettsia and parasites. Such a formulation contains at least one antigen (i.e. a substance which has the ability to combine specifically with the final products of an immune response, i.e. antibodies and/or surface receptors on T-cells) of biological origin. A biological antigen may for example be a live (optionally attenuated) microorganism, a killed microorganism, a toxin of an organism (optionally in an inactivated form, a so called toxoid), or any another metabolite of an organism, a subunit of such an organism such as for example DNA of the organism or a surface molecule of such an organism such as an outer surface protein, outer membrane protein, lipopolysaccharide, carbohydrate etc. In any case, the term “biological antigen” covers a live or killed microorganism, and a biological molecule (preferably a protein or polysaccharide) derived from an organism such as a bacterium, virus, animal, protist, fungus etc. The term “derived from” encompasses that the biological molecule itself or a precursor thereof is produced by the organism.

The amount of interaction between the antigen present in an antigenic formulation and antibodies or T-cells depends not only on the intrinsic binding property of the antigen with the respective antibodies or receptors of T-cells, but also on the availability of the relevant binding site(s), also called epitope(s), of the antigen in the formulation. This availability depends on its turn on the presentation, and preservation, of the antigen in the formulation. Ideal formulations provide an optimal presentation of antigens and preferably, protect the antigen against degradation. In the art, formulations comprising inert oils such as light hydrocarbon oil (e.g. Marcol 52™) and squalane are used the most for antigen presentation. For human applications vitamin E acetate is widely used.

Still there is a need for alterative formulations which provide good antigen presentation. To this end, an antigenic formulation according to the preamble has been devised, wherein the formulation comprises an oil containing as a principle constituent (typically more than 50% on a weight basis of the oil itself) a fatty acid ester of eleostearic acid. Surprisingly it has been found that the availability of various different types of antigens in the present formulation for combination with antibodies, is improved over prior art formulations and also, that the presentation may be even further improved by subjecting the formulation to ultra violet radiation. The latter is in complete contradiction with what one expects when subjecting a formulation containing an antigen with UV radiation. As is commonly known, UV radiation is detrimental for the constitution of many antigens, in particular for proteins. The reason for the found improved presentation of the antigens is unclear. A concomitant advantage of the use of a fatty acid of eleostearic acid is its biodegradability due to which the oil may leave less traces in the body when compared to for example non biodegradable oils such as light hydrocarbon oil and squalane. The present invention also pertains to the use of an oil containing as a principle constituent a fatty acid ester of eleostearic acid to manufacture an antigenic formulation comprising at least one biological antigen.

It is noted that the oil may be used to constitute an emulsion in conjunction with an aqueous phase. As is commonly known, an emulsion is a dispersed system containing at least two immiscible liquid phases. Invariably, one of the two immiscible phases is aqueous while the other is an oil (which simply denotes a water-immiscible liquid). Whether the aqueous or the oil phase becomes the dispersed phase depends i.a. on the amounts of the two liquid phases present. An emulsion in which the oil is dispersed as droplets throughout the aqueous phase is termed an oil-in-water (O/W) emulsion. When water is the dispersed phase and the oil is the dispersing medium (also called continuous medium), the emulsion is of the water-in-oil (W/O) type. Also, multiple emulsions have been developed, in particular with a view to delaying the release of an active ingredient. In these types of emulsion three (or more) phases are present. Such an emulsion is for example of the W/O/W or O/W/O type. It is known that biological antigens may give rise to a faster and better immunological response when emulsified instead of being simply suspended in an aqueous formulation.

The fatty acid ester may be a triglyceride of eleostearic acid, which is a common form of naturally occurring esters of eleostearic acid. Such oils preferably contain between 60 and 90% of the fatty acid ester of eleostearic acid. A typical oil that can be used in the present invention is derived from Aleurites fordii seed oil (also called “tung oil” or “Chinese wood oil”). This seed oil is commonly available and inexpensive. A tuned derivative oil (to meet specific requirements) can for example be made from this naturally occurring oil by removing impurities from the actual seed oil or by adding components such as viscosity modifiers, stabilisers, detergents etc. In an embodiment the oil is actual tung oil, i.e. the oil obtained from the tung tree. Tung oil contains over 50% (w/w) of an ester of eleostearic acid, typically between 73 and 82% (w/w) which makes it ideally suitable for use in formulations according to the present invention.

In an embodiment the formulation is an emulsion of the oil in an aqueous liquid. Preferably, the antigen is present in the aqueous liquid. Examples of suitable antigens are APX III toxin, antigens of bovine respiratory syncytial virus (such as a subunit of this virus or the virus as a whole) and antigen of parainfluenza 3 virus (such as a subunit of this virus or the virus as a whole).

In yet another embodiment the formulation is exposed to UV radiation. As a complete surprise it was found that the antigen presentation may be even further improved by subjecting the formulation to ultra violet radiation.

The invention will be further explained in the following examples.

Example 1 RTX toxin APXIII formulated in an oil in water emulsion.

Example 2 Inactivated BRS virus formulated in an oil in water emulsion.

Example 3 Inactivated PI-3 virus formulated in an oil in water emulsion.

Example 4 RTX toxin APXIII formulated in an alternative oil in water emulsion.

### EXAMPLE 1

As a first example formulations were made with the bacterial repeat-in-toxin APX III as produced by the pig pathogen Actinobacillus pleurpneumoniae (also called APP) This antigen is used as a vaccine component in the commercially available vaccine Porcilis APPTM (available from Intervet Schering-Plough Animal Health, Boxmeer, The Netherlands) and can be used in a diagnostic test to establish whether or not a pig has been infected by any of the APP serotypes 2, 3, 4, 6 or 8 (which serotypes produce AxpIII).

A solution of Tween 80 (Uniqema Nederland BV, Gouda, The Netherlands) in 0.01 M phosphate buffer (PBS) was made by magnetically mixing 40.79 grams of the Tween 80 in 159.21 grams of the PBS buffer (50° C.) until a clear solution was obtained. This solution was cooled to about 8° C. To 53.07 grams of this solution 50 grams tung oil (Vliegenthart B V, Tiel, The Netherlands) having a temperature of about 20° C. was slowly added and mixed under a nitrogen atmosphere, using an 14N rotor/stator ultra turrax mixer (IKA, Staufen, Germany). The addition of the oil was accomplished in 3 minutes and 12 seconds at a mixing speed of about 11000 rpm. Thereafter the emulsion was stirred for another 10 minutes at about 22000 rpm. During this process the temperature of the emulsion rose from 16° C. to 32° C. It appeared that 80% of the oil droplets had a diameter below 2 μm (determined microscopically). To 9.25 grams of the emulsion, 50.58 grams of an aqueous (0.01 M PBS) suspension of APXIII antigen was added to reach the final APXIII formulation. The antigen concentration aimed at in this final formulation was 25 U/ml.

To constitute a control formulation, 50.58 grams of the same APXIII antigen suspension was added to 9.25 grams of 0.01 M PBS (thus also aiming at a concentration of APXIII of 25 U/ml).

The titre of the formulations was determined by an antigenic mass ELISA, using antibodies directed against APX III. In order to establish whether or not the formulations may preserve the toxin from degradation, a test was performed wherein the formulations were exposed to ultra violet radiation. In this test, the Mirasol PRT system (Illuminator version 5.1, available from CaridianBCT, Lakewood, Colo., USA) was used. About 57 ml of a sample to be exposed was transferred to a plastic bag that comes standard with the Mirasol equipment. After that, 120 ml oxygen gas was added as well. The bag including its content was positioned in the Mirasol equipment in line with manufacturer's instructions and the (standard) UV-irradiation program was performed. The total irradiation time was 15 minutes. After the UV treatment the oxygen gas was removed by flushing with nitrogen gas.

The titre as determined in the formulations is depicted in Table 1. The titres are the average value of two measurements.

It is apparent that the oil has a positive effect on the presentation of the antigen in de ELISA test: with the oil the titre increases 13 units/ml. It appears that the availability of the relevant binding site of APXIII is increased due to the presence of the tung oil. Remarkable is the increase in titre due to the exposure with UV radiation from 43 to 69 U/ml.

TABLE 1
APXIII titres of various formulations,
either with or without UV exposure

| test no | sample description | UV | titre [U/ml] |
|---|---|---|---|
| 1 | APXIII in water, control | no | 30 |
| 2 | APXIII in oil-in-water emulsion | no | 43 |
| 3 | APXIII in water, control | yes | 30 |
| 4 | APXIII in oil-in-water emulsion | yes | 69 |

### EXAMPLE 2

In a second example formulations were made with inactivated BRS virus. This antigen is used as a vaccine component in the commercially available vaccine Bovilis Bovipast™ (available from Intervet Schering-Plough Animal Health, Boxmeer, The Netherlands) and can be used in a diagnostic test to establish whether or not cattle has been infected with the bovine respiratory syncytial virus.

A solution of Tween 80 (ICI Americas, Wilmington Del.) in 0.01 M phosphate buffer (PBS) was made by magnetically mixing 40.79 grams of the Tween 80 in 159.21 grams of the PBS buffer (50° C.) until a clear solution was obtained. This solution was cooled to about 20° C. To 53.07 grams of this solution 50 grams tung oil (Vliegenthart BV, Tiel, The Netherlands) having a temperature of about 20° C. was slowly added and mixed under a nitrogen atmosphere, using an 18N rotor/stator ultra turrax mixer (IKA, Staufen, Germany). The addition of the oil was accomplished in 3 minutes and 13 seconds at a mixing speed of 17000 rpm. Thereafter the emulsion was stirred for 5 minutes and 30 seconds at 20200 rpm and another 3 minutes and 30 seconds at 24000 rpm. During this process the temperature of the emulsion rose from 22° C. to 35° C. It appeared that about 80-90% of the oil droplets had a diameter below 5 μm (determined microscopically).

To 9.25 grams of the emulsion, 50.58 grams of an aqueous (0.01 M PBS) suspension of inactivated BRS virus was added to reach the final formulation. The antigen concentration aimed at in this final formulation was 125 U/ml.

To constitute a control formulation, 50.58 grams of the same BRS virus suspension was added to 9.25 grams of 0.01 M PBS (thus also aiming at a concentration of BRSV of 125 U/ml).

The titre of the formulations was determined by an antigenic mass ELISA, using antibodies directed against BRS virus. Also, a test was performed wherein the formulations were exposed to ultra violet radiation. In this test, the Mirasol PRT system (Illuminator version 5.1, available from CaridianBCT, Lakewood, Colo., USA) was used. About 57 ml of a sample to be exposed was transferred to a plastic bag that comes standard with the Mirasol equipment. After that, 120 ml oxygen gas was added as well. The bag including its content was positioned in the Mirasol equipment in line with manufacturer's instructions and the (standard) UV-irradiation program was performed. The total irradiation time was 15 minutes. After the UV treatment the oxygen gas was removed by flushing with nitrogen gas.

The titre as determined in the formulations is depicted in Table 2. The titres are the average value of two formulations, each measured twice.

TABLE 2
BRSV titres of various formulations,
either with or without UV exposure

| test no | sample description | UV | titre [U/ml] |
|---|---|---|---|
| 1 | BRSV in water, control | no | 125 |
| 2 | BRSV in oil-in-water emulsion | no | 147 |
| 3 | BRSV in water, control | yes | 0 |
| 4 | BRSV in oil-in-water emulsion | yes | 147 |

It is apparent that the oil has a positive effect on the presentation of the antigen in de ELISA test: with the oil the titre increases 22 units/ml. It appears that the availability of the relevant binding site of BRSV is increased due to the presence of the tung oil. Also remarkable is the apparent full protection against exposure with UV radiation. Without the presence of the tung oil, the UV radiation results in a complete loss of titre. With the tung oil, the titre remains constant.

### EXAMPLE 3

In a third example formulations were made with inactivated parainfluenza-3 virus. This antigen is used as a vaccine component in the commercially available vaccine Bovilis Bovipast™ (available from Intervet Schering-Plough Animal Health, Boxmeer, The Netherlands) and can be used in a diagnostic test to establish whether or not cattle has been infected with the parainfluenza virus.

The same oil emulsion as made for preparation of the BRSV formulation was used. To 9.25 grams of the emulsion 50.58 grams of an aqueous (0.01 M PBS) suspension of inactivated PI-3 virus was added to reach the final formulation. The antigen concentration aimed at in this final formulation was 25 U/ml.

To constitute a control formulation, 50.58 grams of the same PI-3 suspension was added to 9.25 grams of 0.01 M PBS (thus also aiming at a concentration of PI-3 of 25 U/ml).

The titer of the formulations was determined by an antigenic mass ELISA, using antibodies directed against PI-3 virus. Also, a test was performed wherein the formulations were exposed to ultra violet radiation. In this test, the Mirasol PRT system (Illuminator version 5.1, available from CaridianBCT, Lakewood, Colo., USA) was used. About 57 ml of a sample to be exposed was transferred to a plastic bag that comes standard with the Mirasol equipment. After that, 120 ml oxygen gas was added as well. The bag including its content was positioned in the Mirasol equipment in line with manufacturer's instructions and the (standard) UV-irradiation program was performed. The total irradiation time was 15 minutes. After the UV treatment the oxygen gas was removed by flushing with nitrogen gas.

The titre as determined in the formulations is depicted in Table 3. The titres are the average value of two formulations, each measured twice.

TABLE 3
PI-3 titres of various formulations,
either with or without UV exposure

| test no | sample description | UV | titre [U/ml] |
|---|---|---|---|
| 1 | PI-3 in water, control | no | 26 |
| 2 | PI-3 in oil-in-water emulsion | no | 28 |
| 3 | PI-3 in water, control | yes | 0 |
| 4 | PI-3 in oil-in-water emulsion | yes | 34 |

It seems that the oil has a positive effect on the presentation of the antigen in de ELISA test: with the oil the titre increases 2 units/ml (although the standard deviations on the results in test 1 and 2, viz. 0.9 and 1.1 respectively, are such that statistically this increase may not be relevant). Very remarkable is the increase in titre after exposure with UV radiation, even more so since without the presence of the tung oil, the UV radiation results in a complete loss of titre. With the tung oil, having as a main constituent a fatty acid ester of eleostearic acid, the titre increases.

### EXAMPLE 4

In this example the experiment of Example 1 was repeated with an alternative fatty ester of eleostearic acid, viz. the methyl ester of eleostearic acid. This ester was used in pure form instead of the tung oil as described in Example 1.

The resultant formulation was subjected to the same titre determination as described in Example 1, including an UV treatment as described. The difference being that only 1 minute of UV treatment was applied given the fact that 250 μl of the resultant formulation was UV treated instead of the 57 ml as described in Example 1. The results are indicated below in Table 4.

TABLE 4
APXIII titres of various formulations,
either with or without UV exposure

| test no | sample description | UV | titre [U/ml] |
|---|---|---|---|
| 1 | APXIII in water, control | no | 24 |
| 2 | APXIII in oil-in-water emulsion | no | 32 |
| 3 | APXIII in oil-in-water emulsion | yes | 42 |

It is apparent that the methyl ester of eleostearic acid has a positive effect on the presentation of the antigen in de ELISA test: with the oil the titre increases 8 units/ml. It appears that the availability of the relevant binding site of APXIII is increased due to the presence of the oil. Again a remarkable increase in titre is provided (from 32 to 42 U/ml) due to the exposure with UV radiation.

1. An antigenic formulation comprising a biological antigen, characterised in that the formulation comprises an oil containing as a principle constituent a fatty acid ester of eleostearic acid.

2. The formulation of claim 1, characterised in that the ester is a triglyceride of eleostearic acid.

3. The formulation of claim 2, characterised in that the oil comprises between 60 and 90% (w/w) of the fatty acid ester of eleostearic acid.

4. The formulation of claim 1, characterised in that the oil is derived from Aleurites fordii seed oil.

5. The formulation of claim 4, characterised in that the oil is tung oil.

6. The formulation of claim 1, characterised in that the formulation is an emulsion of the oil in an aqueous liquid.

7. The formulation of claim 6, characterised in that the antigen is present in the aqueous liquid.

8. The formulation of claim 7, characterised in that the antigen is chosen from APX III toxin, bovine respiratory syncytial virus antigen and parainfluenza 3 virus antigen.

9. The formulation of claim 1, characterised in that the formulation is exposed to UV radiation.

10. (canceled)

11. The formulation of claim 1, characterised in that the oil comprises between 60 and 90% (w/w) of the fatty acid ester of eleostearic acid.

12. The formulation of claim 2, characterised in that the oil is derived from Aleurites fordii seed oil.

13. The formulation of claim 3, characterised in that the oil is derived from Aleurites fordii seed oil.

14. The formulation of claim 13, characterised in that the oil is tung oil.

15. The formulation of claim 14, characterised in that the formulation is an emulsion of the oil in an aqueous liquid.

16. The formulation of claim 15, characterised in that the antigen is present in the aqueous liquid.

17. The formulation of claim 16, characterised in that the antigen is chosen from APX III toxin, bovine respiratory syncytial virus antigen and parainfluenza 3 virus antigen.

18. The formulation of claim 17, characterised in that the formulation is exposed to UV radiation.

19. The formulation of claim 5, characterised in that the formulation is an emulsion of the oil in an aqueous liquid.

20. The formulation of claim 19, characterised in that the antigen is chosen from APX III toxin, bovine respiratory syncytial virus antigen and parainfluenza 3 virus antigen.

21. The formulation of claim 20, characterised in that the formulation is exposed to UV radiation.
